(19) United States
(12) Patent Application Publication
(10) Pub. No.: US 2011/0009703 A1
(43) Pub. Date: Jan. 13, 2011

(54) FLEXIBLE EXTERNAL CANNULA SHEATH

(76) Inventor: Robert C. Smith, Middletown, CT (US)

Correspondence Address: Tyco Healthcare Group LP;d/b/a Covidien, 555 Long Wharf Drive, Mail Stop 8-N1, Legal Department, New Haven, CT, 06511, US

(21) Appl. No.: 12/526,047

(22) PCT Filed: Feb. 15, 2008
(86) PCT No.: PCT/US08/02074

§ 371 Date: Aug. 6, 2009

Related U.S. Application Data
(60) Provisional application No. 60/902,411, filed on Feb. 20, 2007.

(51) Int. Cl. A61B 1/32

(52) U.S. Cl. 600/203

(57) ABSTRACT

A flexible external cannula sheath assembly includes a tubular member having a proximal end portion and a distal end portion and a seal positioned adjacent the distal end portion of the tubular member. The seal defines a hole in a central portion thereof for receiving surgical instruments therethrough. The flexible sheath is formed of a flexible material and is configured and dimensioned to surround an external surface of a cannula. The seal is displaced a predetermined distance from a distal end of the cannula to provide additional flexibility to the assembly.

### BACKGROUND

1. Field of the Disclosure

The present disclosure relates to a flexible external cannula sheath including a sealing device. More particularly, the present disclosure relates to a cannula sheath for use in laparoscopic surgical procedures including a sealing member, which seals a cannula sheath lumen upon insertion of an instrument through the cannula.

2. Background of Related Art

Surgical procedures have been developed during which surgical instruments are passed through small openings in body tissue to access internal surgical sites. These surgical procedures, commonly referred to as endoscopic procedures, have become widely accepted. The term endoscopic as used herein is defined to include all types of minimally invasive surgical procedures including laparoscopic and arthroscopic procedures. Typically, during these procedures, after an incision has been formed in the body tissue, a cannula or cannula sheath defining a lumen is inserted through the incision and fixedly positioned in relation to the surgical site. During some such procedures, the body cavity is inflated with an insufflation gas to create a working area inside a patient and allow a trocar to penetrate a body cavity without the risk of damaging underlying organs within the body cavity. Generally, the cannula includes a sealing member or members to seal the cannula lumen prior to and after insertion of a surgical instrument into the body cavity to prevent insufflation gases within the body cavity from escaping. The sealing member or members often include adjustable sealing elements capable of sealing about multiple instruments of different sizes and shapes.

Additionally, in view of the widespread acceptance of endoscopic procedures in surgery, numerous endoscopic instruments have been developed which allow the surgeon to perform complex surgical procedures with minimal incision into the skin and tissue surrounding a particular body cavity or anatomical region. Many of these endoscopic instruments are rigid and in a fixed position. However, an increasing number of endoscopic instruments incorporate the ability to flex during insertion into the lumen defined by the cannula sheath. For example, surgical procedures such as angioplasty take advantage of the benefits provided by a flexible introducer sheath. One benefit which is provided by a flexible introducer sheath is that the sheath serves to protect the skin puncture site.

Although flexible cannula sheaths that adequately perform the intended functions are known, improvements to the known devices are warranted. For example, it is common practice to have a sealing member mounted in a proximal housing portion of the cannula. Generally, this configuration adequately performs the intended function of preventing the escape of insufflation gases. However, having the sealing member in the proximal housing portion of the cannula requires the housing to be relatively large.

Thus, a continuing need exists for a flexible self-sealing cannula sheath which is compact in size and allows for easy insertion and removal of multiple size instruments into and from the cannula sheath.

### SUMMARY

The present disclosure provides a novel flexible self-sealing cannula sheath assembly which is compact in size and allows for easy insertion and removal of multiple size instruments into and from the cannula sheath. The flexible self-sealing cannula sheath assembly includes a flexible sheath which is configured and dimensioned to enclose a cannula. The flexible sheath includes an instrument seal on a distal end thereof. The flexibility of the sheath and seal permit surgical personnel to maneuver surgical instruments within the cannula during endoscopic procedures, without compromising the integrity of the seal.

In accordance with an embodiment of the present disclosure, a flexible external cannula sheath is provided which includes a tubular member having a proximal end portion and a distal end portion and an instrument seal positioned adjacent the distal end portion of the tubular member. The seal defines a hole in a central portion thereof for receiving surgical instruments therethrough. The flexible sheath is formed of a resilient flexible material and is configured and dimensioned to surround an external surface of a cannula. The flexibility of the sheath, combined with the fact that the seal is displaced a predetermined distance from a distal end of the cannula, provides the surgical personnel with maneuverability advantages when utilizing the apparatus.

The flexible external cannula sheath may also include a housing positioned adjacent and proximal to the proximal end portion of the tubular member. A zero-seal is preferably mounted within the housing. A guide member is attached to a distal end of the tubular member to facilitate easy insertion of the flexible cannula sheath into the patient.

In another embodiment of the present disclosure, a self-sealing cannula and sheath assembly is provided including a cannula body having a proximal end portion and a distal end portion, and a flexible sheath superposed at least a portion of the cannula body, wherein the flexible sheath includes an instrument seal mounted on a distal portion thereof. Each of the cannula body and the flexible sheath define a longitudinal lumen for removably receiving surgical instruments therein. The proximal end portion of the cannula body defines an inlet opening and the distal end portion defines an outlet opening.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiments given below, serve to explain the principles of the disclosure.

FIG. 1 is a side cross-sectional view of a self-sealing cannula and sheath assembly in accordance with an embodiment of the present disclosure;

FIG. 2 is a perspective view of a self-sealing cannula sheath in accordance with an embodiment of the present disclosure;

FIG. 3 is a partial perspective view of a self-sealing cannula sheath in accordance with another embodiment of the present disclosure;

FIG. 4 is an exploded perspective view of a self-sealing cannula and sheath assembly in accordance with an embodiment of the present disclosure;

FIG. 5 is a perspective view of a self-sealing cannula sheath in accordance with an embodiment of the present disclosure;

FIG. 6 is a side cross-sectional view of a self-sealing cannula and sheath assembly having an instrument inserted therein, in accordance with an embodiment of the present disclosure; and

FIG. 7 is a side cross-sectional view of a self-sealing cannula and sheath assembly having an instrument inserted therein, in accordance with an embodiment of the present disclosure.

### DETAILED DESCRIPTION OF PREFERRED EMBODIMENTS

Preferred embodiments of the presently disclosed self-sealing cannula sheath will now be described in detail with reference to the figures, in which like reference numerals identify corresponding elements throughout the several views.

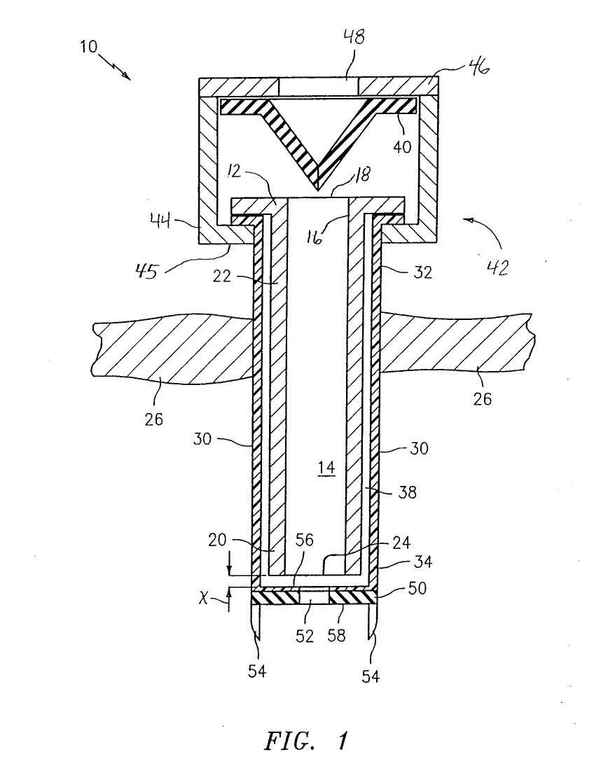

A self-sealing cannula and sheath assembly, shown generally as reference numeral 10 in FIG. 1, includes a cannula body 12 and a sheath 30, each of which define a longitudinal lumen 14. The cannula body includes a proximal end portion 16, which preferably defines an inlet opening 18, a central body portion 22, which is preferably cylindrical, and a distal end portion 20, which defines an outlet opening 24.

Self-sealing cannula and sheath assembly 10 also includes a proximal housing portion 42. Proximal housing portion 42 includes a cylindrical member 44 and a cover 46. The cylindrical member has a shoulder 45 formed on the distal end thereof. The shoulder 45 defines an opening for receiving sheath 30. Furthermore, a shoulder on the proximal end of sheath 30 engages shoulder 45. Cover 46 defines an inlet opening 48 for receiving surgical instruments into the self-sealing cannula and sheath assembly 10. Cover 46 engages the proximal end of cylindrical member 44 and may be attached thereto by methods known to one having ordinary skill in the art. Although self-sealing cannula and sheath assembly 10 is illustrated as being formed of multiple components, e.g., proximal housing portion 42 is formed separately from the cannula body 12, it is envisioned that self-sealing cannula and sheath assembly 10, or any portion thereof, may be of monolithic construction.

Flexible sheath 30 having proximal and distal end portions 32 and 34 is secured at the proximal end portion 32 between an inner surface of a shoulder formed on the proximal end of central body portion 22 and an inner surface of shoulder 45. Proximal end portion 32 is secured in a manner that will form a seal between the shoulder formed on the proximal end of central body portion 22 and an inner surface of shoulder 45. The distal end portion 34 of flexible sheath 30 extends distally beyond the distal end portion 20 of the cannula body 12.

An instrument seal 50 having a proximal surface 56 and a distal surface 58 is connected to the distal end portion 34 of the flexible sheath 30. Seal 50 defines an opening 52 in the central region of the seal. As discussed above, the flexible sheath 30 extends distally beyond the distal end portion 20 of the cannula body 12. Accordingly, a gap having a predetermined width x is formed between the end of distal end portion 20 and the proximal surface 56 of seal 50. The purpose of the gap x is ensure that sheath 30 and seal 50 are free to move relative to cannula body 12. Therefore, surgical personnel will have the ability to maneuver the instruments within the cannula without compromising the effectiveness of seal 50. The dimensions of the gap x are preferably in the range of 0.1 cm to 10 cm.

An annular pocket 38 is defined between an outer surface of central body portion 22 and an inner surface of flexible sheath 30. The annular pocket 38 becomes pressurized by the insufflation gas which enters into the lumen 14 through the opening 52 in seal 50. The pressure against the inside surface of flexible sheath 30 maintains a seal between the outer surface of flexible sheath 30 and the inner surface of the dermis 26 of the patient. Therefore, the loss of insufflation gas around the circumference of the cannula assembly is effectively prevented. An access port may be defined in flexible sheath 30 to communicate directly with annular pocket 38 and to regulate the pressure within the annular pocket. The annular pocket 38 also provides the cannula with a buffer zone which allows movement of the cannula and instruments inserted within the cannula. Flexible sheath 30 is formed from natural materials, synthetic materials or a combination of natural and synthetic materials and is preferably formed of a fabric/elastomer material.

A flexible zero-seal 40 for sealing cannula lumen 14 to prevent or minimize the loss of gasses through inlet opening 48 is provided within proximal housing portion 42. Flexible zero-seal 40 is designed to provide a positive seal when there is no instrument positioned therein. Flexible zero-seal 40 may be a flexible membrane having an expandable slit formed therein. Alternately, other types of seals may be used in place of zero-seal 40, or one or more additional seals may be installed adjacent proximal housing portion 42.

In use, a body incision or hole is typically made through the dermis 26 of the patient with a trocar. Self-sealing cannula and sheath assembly 10 is then positioned through the body incision into an insufflated cavity. At least one guide member 54 is attached to a distal end of flexible sheath 30 to facilitate easy entry of the cannula assembly 10 into the body incision. It is preferred that the distal end of guide member 54 is beveled as illustrated. Pressurized gas from within the cavity flows into annular pocket 38 via opening 52 in seal 50, or via an access port, effectively inflating flexible sheath 30. Thereafter, when an instrument is inserted through lumen 14 and through seal 50, restricting flow in or out of annular pocket 38, annular pocket 38 will remain pressurized. To ensure that the pressurized gas in the insufflated cavity does not escape upon the entry of an instrument through the lumen 14, flexible seal 40 is preferably a zero seal which will seal uniformly around the body of the surgical instrument.

In one preferred embodiment, a synthetic material is used to form zero-seal 40. Cannula and sheath assembly 10, according to the present disclosure, by virtue of zero-seal 40, is primed for self-sealing when it is disposed in an insufflated body cavity. However, depending on, inter alia, materials of construction or configuration, it is possible that when a surgical instrument is inserted through lumen 14, zero-seal 40 will not compress uniformly about the surgical instrument thereby creating a number of gaps between zero-seal 40 and the surgical instrument. In prior art devices, these gaps would allow the pressurized gas in the insufflated cavity to escape from the body cavity, thereby minimizing the effectiveness of the self-sealing cannula. However, in accordance with the present disclosure, zero-seal 40 is formed from a synthetic material that will compress uniformly around the body of a surgical instrument and form a pressure barrier which eliminates or minimizes the gaps. It is preferred that the synthetic material be nylon, Kevlar®, or any other material that will compress uniformly when a surgical instrument is inserted in the cannula body 12. The selected material may also be of knitted construction to minimize or prevent wrinkling of zero-seal 40 when a surgical instrument is inserted into the cannula body. Notwithstanding the advances in the choice of materials and construction of the zero-seal 40, it is likely that at least some insufflation gas will leak out. Accordingly, in accordance with the present disclosure, seal 50 will assist in minimizing or eliminating the amount of gas that is bypassing seal 40.

The selected material will preferably have a low coefficient of friction so that insertion and removal of a surgical instrument does not require excessive amounts of force. An interior surface of flexible sheath 30 may also be coated with a lubricious material to minimize the friction between the cannula body 12 and flexible sheath 30. Although flexible sheath 30 will preferably have a low coefficient of friction, the combination of zero-seal 40 and seal 50 is capable of maintaining the surgical instrument properly positioned within cannula assembly 10 during the laparoscopic procedure. Further still, the selected material is preferably thin yet durable enough to prevent the surgical instrument from inadvertently puncturing membrane 30 during insertion, removal or operation of said instrument.

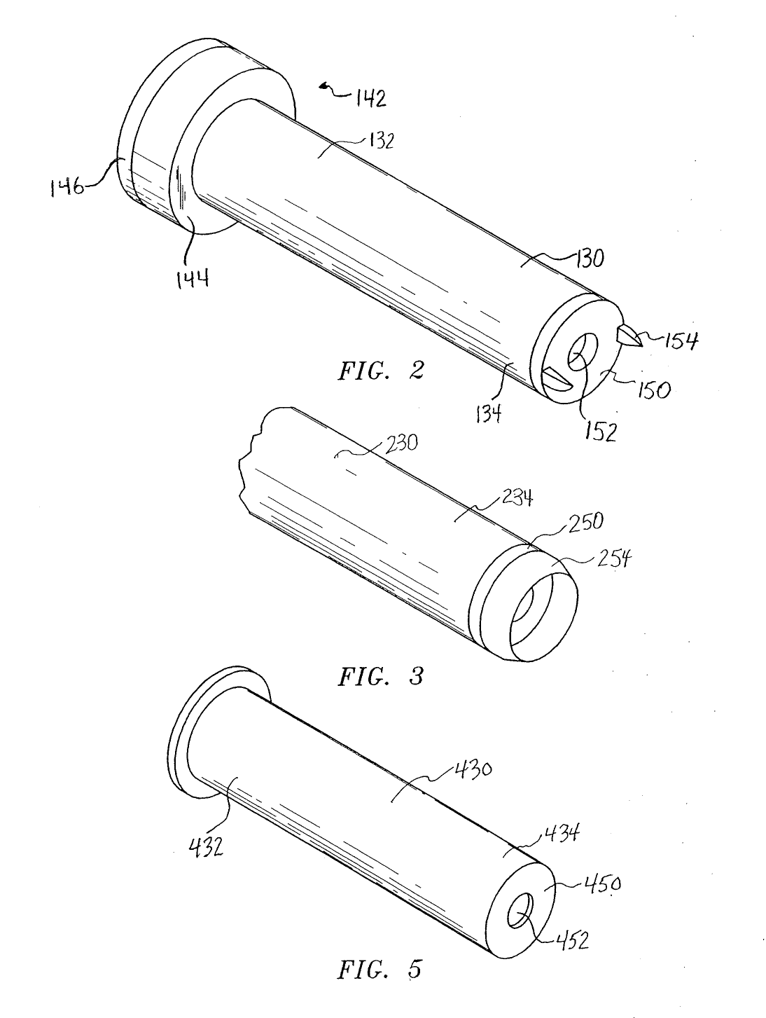

Referring now to FIG. 2, a perspective view of a self-sealing cannula sheath 130 in accordance with an embodiment of the present disclosure is shown. Flexible sheath 130 has a proximal end portion 132 and a distal end portion 134. A housing 142 is mounted on the proximal end portion 132 of sheath 130. Housing 142 includes a cylindrical member 144 and a cover 146. The cylindrical member 144 defines an opening for receiving sheath 130. The distal end portion 134 is inserted through the opening defined by cylindrical member 144. When sheath 130 is positioned within cylindrical member 144, cylindrical member 144 is configured to engage the proximal end of sheath 130. Cover 146 engages the proximal end of cylindrical member 144 and may be attached thereto by methods known to one having ordinary skill in the art.

A seal 150 is connected to the distal end portion 134 of the flexible sheath 130. Seal 150 defines an opening 152 in the central region of the seal. In accordance with the present disclosure, sheath 130 exhibits sufficient flexibility such that surgical personnel will have the ability to maneuver instruments within a cannula positioned within flexible sheath 130 without compromising the integrity and effectiveness of seal 150.

At least one guide member 154 is attached to a distal end of flexible sheath 130 to facilitate easy entry of the cannula assembly 10 into the body incision. It is preferred that the distal ends of guide members 154 are beveled as illustrated.

FIG. 3 is a partial perspective view of another embodiment of a self-sealing cannula sheath 230 in accordance with the present disclosure. More specifically, FIG. 3 illustrates a partial view of a cannula sheath 230 having a guide member 254 attached to a distal end portion 234. As seen by comparing FIGS. 2 and 3, guide member 254 differs from guide members 154 most notably to the extent that guide member 254 is a fully annular member.

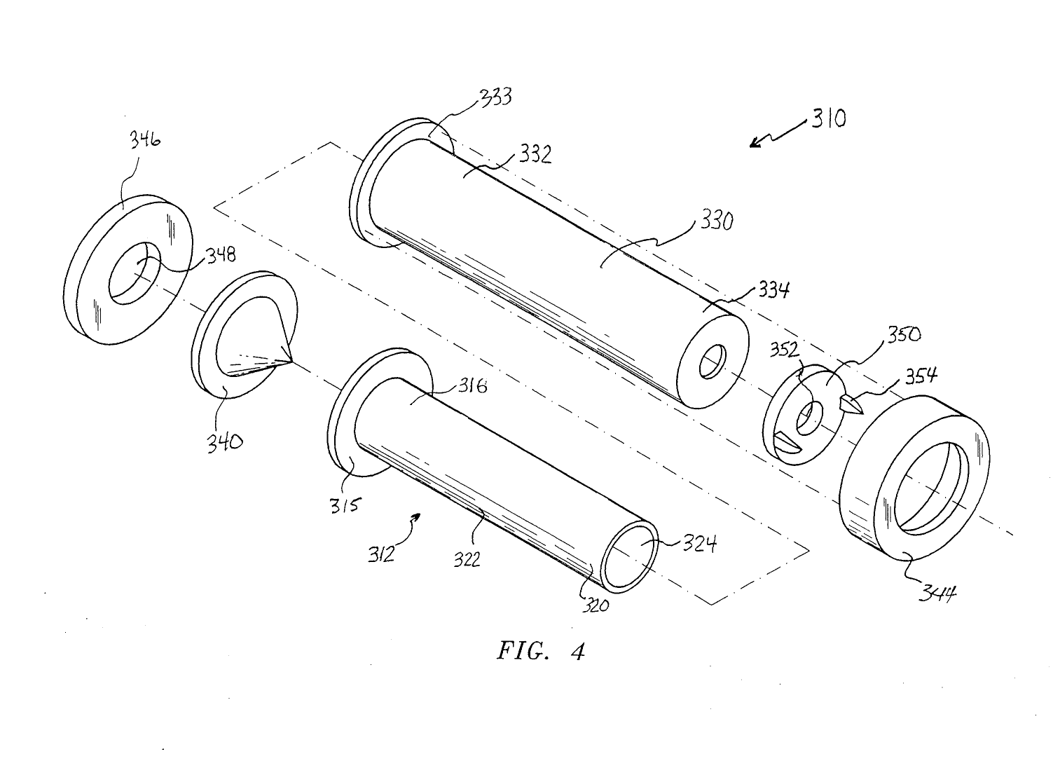

Referring now to FIG. 4, an exploded perspective view of the self-sealing cannula and sheath assembly illustrated in FIG. 1 is shown generally as reference numeral 310. Self-sealing cannula and sheath assembly 310 includes a cannula body 312 and a sheath 330, each of which define a longitudinal lumen (see FIG. 1). The cannula body includes a proximal end portion 316, a central body portion 322, which is preferably cylindrical, and a distal end portion 320, which defines an outlet opening 324.

A flexible zero-seal 340 is positioned adjacent to the proximal end portion 316 of cannula 312. Flexible zero-seal 340 seals uniformly around the body of a surgical instrument, to ensure that pressurized gas in an insufflated cavity does not escape upon the entry of the instrument through the lumen 314.

Flexible sheath 330 has a proximal end portion 332 and a distal end portion 334. The distal end portion 334 of sheath 330 and the distal end portion 320 of cannula 322 are each configured as a cylinder. The diameter of the cylindrical portion of cannula 322 is less than the diameter the cylindrical portion of sheath 330. Accordingly, as indicated by the dashed lines in FIG. 4, the cannula and sheath assembly 310 is assembled by sliding cannula 322 distally into the proximal end portion 332 of sheath 330 until a distal side of an annular ring 315 engages a proximal side of an annular ring 333.

A seal 350 having a proximal surface and a distal surface is connected to the distal end portion 334 of the flexible sheath 330. Seal 350 defines an opening 352 in the central region of the seal for receiving surgical instruments which are inserted through flexible seal 340 and through cannula 322. Therefore, the combination of flexible seal 340 and seal 350 is capable of maintaining the surgical instrument properly positioned within cannula and sheath assembly 310 during the laparoscopic procedure while minimizing or eliminating the loss of insufflation gas.

A guide member 354 is attached to a distal end of flexible sheath 330 to facilitate easy entry of the cannula and sheath assembly 310 into the body incision. It is preferred that the distal end of guide member 354 is beveled as illustrated.

Self-sealing cannula and sheath assembly 310 is held together by the components of a proximal housing portion. More specifically, proximal housing portion includes a cylindrical member 344 and a cover 346. The cylindrical member 344 is configured to fit around flexible sheath 330. Accordingly, cylindrical member 344 is slid in the proximal direction beginning at the distal end portion 334 of the cannula sheath 330. The cylindrical member 344 is moved in the proximal direction until it engages a distal side of annular ring 333. At that point, a cover 346 engages the proximal end of cylindrical member 344 thereby forming the proximal housing portion. Self-sealing cannula and sheath assembly 310 is then ready to be inserted within a body incision or hole made through the dermis 326 of the patient with a trocar. Self-sealing cannula assembly 310 is then positioned through the body incision into an insufflated cavity.

Turning now to FIG. 5, a perspective view of a self-sealing cannula sheath in accordance with another embodiment of the present disclosure is illustrated. More specifically, FIG. 5 illustrates a cannula sheath 430 having a proximal end 432 and a distal end 434. The cannula sheath 430 includes a seal 450 integrated into the distal end thereof. Seal 450 defines a circular opening 452 for receiving and sealing against a surgical instrument when the surgical instrument is inserted through the self-sealing cannula sheath. It is contemplated that guide members, although not shown, may be attached to the distal end of sheath 430 as shown above.

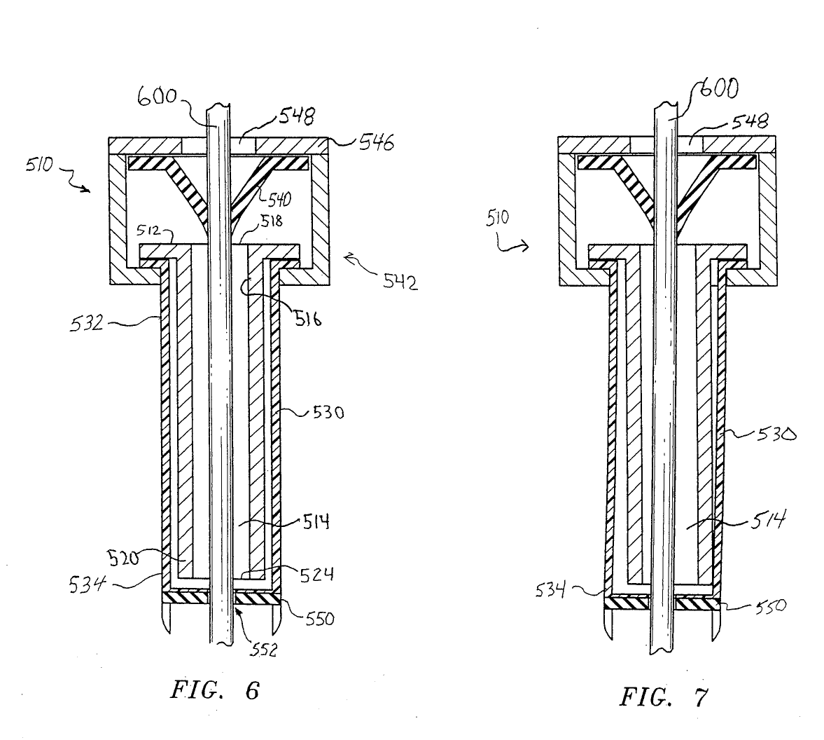

FIGS. 6 and 7 are side cross-sectional views of a self-sealing cannula and sheath assembly 510 with a surgical instrument 600 inserted therein. Self-sealing cannula and sheath assembly 510 includes a cannula body 512 and a sheath 530, each of which define a longitudinal lumen 514. The cannula body includes a proximal end portion 516, which preferably defines an inlet opening 518, and a distal end portion 520, which defines an outlet opening 524.

Self-sealing cannula and sheath assembly 10 also includes a proximal housing portion 542. Proximal housing portion 542 includes a cylindrical member 544 and a cover 546. Cover 546 defines an inlet opening 548 for receiving surgical instruments into the self-sealing cannula and sheath assembly 510.

Flexible sheath 530 has proximal and distal end portions 532 and 534, respectively. A seal 550 is connected to the distal end of the flexible sheath 530. Seal 550 defines an opening 552 in the central region of the seal. The flexible sheath 530 extends distally beyond the distal end portion 520 of the cannula body 512. Accordingly, a gap having a predetermined width is formed between the end of distal end portion 520 and the seal 550. The purpose of the gap is to ensure that sheath 530 and seal 550 are free to move. Therefore, surgical personnel will have the ability to maneuver the instruments within the cannula without compromising the integrity and effectiveness of seal 550.

A flexible zero-seal 540 for sealing cannula lumen 514 to prevent or minimize the loss of gasses through inlet opening 548 is provided within proximal housing portion 542. Flexible zero-seal 540 is designed to provide a positive seal whether or not an instrument is positioned therein. Flexible zero-seal 540 may be a flexible membrane having an expandable slit formed therein.

In use, a body incision or hole is typically made through the dermis of the patient with a trocar. Self-sealing cannula and sheath assembly 510 is then positioned through the body incision into an insufflated cavity. An instrument 600 is then inserted through opening 548, through zero-seal 540, into lumen 514, through the opening 552 in seal 550 and into the insufflated cavity.

During use of the instrument 600, the surgeon is required to maneuver the instrument 600 in a plurality of positions to complete the surgical procedure. Flexible sheath 530 is designed to accommodate at least a portion of the displacement caused by the instrument 600 being maneuvered by the surgeon. FIG. 6 illustrates the instrument 600 positioned within the cannula and sheath assembly 510 in the substantially vertical position. FIG. 7 illustrates the instrument 600 positioned within the cannula and sheath assembly 510 where in the instrument 600 is displaced from the vertical position. Due to the close proximity between seal 550 and instrument 600, as well as the connection between seal 550 and flexible sheath 530, as instrument 600 is displaced from the vertical axis, flexible sheath 530 is displaced a corresponding amount. As illustrated in FIG. 7, the distal end portion of instrument 600 is displaced to the left. The distal end portion 534 of flexible sheath 530 is also shifted to the left. As shown in the figure, the right side of the distal end portion 534 of flexible sheath 530 is shifted to the left and is touching the distal end portion 520 of cannula body 512, without compromising the integrity of seal 550.

It will be understood that various modifications may be made to the embodiments disclosed herein. For example, the cannula may have a variety of different shapes other than cylindrical, e.g., square, oval, rectangular, etc. Also, the flexible sheath can be fastened to the cannula using any known technique including those not disclosed herein. Therefore, the above description should not be construed as limiting, but merely as exemplifications of preferred embodiments. Those skilled in the art will envision other modifications within the scope and spirit of the following claims.

What is claimed is:

1. A flexible external cannula sheath, the sheath comprising:
a tubular member having a proximal end portion and a distal end portion, wherein the tubular member is formed of a flexible material and is configured and dimensioned to surround an external surface of a cannula; and
a seal positioned adjacent the distal end portion of the tubular member.

2. The flexible external cannula sheath as recited in claim 1 wherein the tubular member is dimensioned and configured to define an annular pocket between an inner surface of the tubular member and an outer surface of the cannula.

3. The flexible external cannula sheath as recited in claim 1 wherein the seal defines a hole in a central portion thereof for receiving surgical instruments therethrough.

4. The flexible external cannula sheath as recited in claim 1 wherein the seal is displaced a predetermined distance from a distal end of the cannula.

5. The flexible external cannula sheath as recited in claim 1 wherein the cannula and the sheath are substantially cylindrical in shape.

6. The flexible external cannula sheath as recited in claim 1 wherein the tubular member is connected to the cannula at a proximal end thereof.

7. The flexible external cannula sheath as recited in claim 6 wherein a seal is formed at the connection between the tubular member and the cannula.

8. The flexible external cannula sheath as recited in claim 1 further comprising a housing positioned adjacent and proximal to the proximal end portion of the tubular member.

9. The flexible external cannula sheath as recited in claim 8 further comprising a seal mounted in the housing.

10. The flexible external cannula sheath as recited in claim 9 wherein the seal mounted in the housing is a zero-seal.

11. The flexible external cannula sheath as recited in claim 1 further comprising a guide member attached to a distal end of the tubular member.

12. The flexible external cannula sheath as recited in claim 11 wherein the distal end of the guide member is beveled.

13. The flexible external cannula sheath as recited in claim 1 wherein the seal positioned adjacent the distal end portion of the tubular member is an instrument seal.

14. A self-sealing cannula and sheath assembly comprising:
a cannula body having a proximal end portion and a distal end portion; and
a flexible sheath superposed at least a portion of the cannula body, wherein the flexible sheath comprises a seal mounted on a distal portion thereof.

15. The self-sealing cannula and sheath assembly as recited in claim 14 wherein each of the cannula body and the flexible sheath define a longitudinal lumen.

16. The self-sealing cannula and sheath assembly as recited in claim 14 wherein the proximal end portion of the cannula body defines an inlet opening and the distal end portion defines an outlet opening.

17. The self-sealing cannula and sheath assembly as recited in claim 14 further comprising a proximal housing portion.

18. The self-sealing cannula and sheath assembly as recited in claim 17 wherein proximal housing portion comprises a cylindrical member and a cover mounted on the cylindrical member.

19. The self-sealing cannula and sheath assembly as recited in claim 18 wherein the cover defines an inlet opening for receiving at least one surgical instrument into the self-sealing cannula and sheath assembly.

20. The self-sealing cannula and sheath assembly as recited in claim 17 further comprising a zero-seal mounted within the proximal housing portion.

21. The self-sealing cannula and sheath assembly as recited in claim 14 wherein the seal defines an opening in a central region of the seal.
